SEQUENCE LISTING (2 sequences)

SEQ ID NO: 1 - DNA, 18 bp, Artificial Sequence
  Description of Artificial Sequence  Synthetic Primer
ggttcggtacttctgact

SEQ ID NO: 2 - DNA, 18 bp, Artificial Sequence
  Description of Artificial Sequence  Synthetic Primer
tggttttgacttgttctg

(19) United States
(12) Patent Application Publication
(10) Pub. No.: US 2006/0153808 A1
(43) Pub. Date: Jul. 13, 2006

(54) Cancer immunotherapy incorporating p53

(75) Inventors: Massimo Cristofanilli, Pearland, TX (US); Savitri Krishnamurthy, Houston, TX (US); Kerstin Menander, Bellaire, TX (US); Gabriel N. Hortobagyi, Bellaire, TX (US)

Correspondence Address: FULBRIGHT & JAWORSKI L.L.P., 600 CONGRESS AVE., SUITE 2400, AUSTIN, TX, 78701, US

(73) Assignees: BOARD OF REGENTS, THE UNIVERSTIY OF TEXAS SYSTEM; Introgen Therapeutics, Inc.

(21) Appl. No.: 11/281,922

(22) Filed: Nov. 17, 2005

Related U.S. Application Data
(60) Provisional application No. 60/628,990, filed on Nov. 17, 2004.

(51) Int. Cl. A61K 48/00

(52) U.S. Cl. 424/93.2

(57) ABSTRACT

A method of stimulating an immune response to a tumor in an immunocompetent subject by administering a p53 expression construct to a tumor. The construct expresses p53 in tumor cells in an amount sufficient to stimulate an immune response against the tumor. Both viral and non-viral delivery systems are contemplated. The method can be combined with chemotherapy agents as well as with other cancer therapies.

This application claims the benefit of the filing date of U.S. provisional patent application Ser. No. 60/628,990, filed Nov. 17, 2004, the entire content of which is hereby incorporated by reference.

### BACKGROUND OF THE INVENTION

I. Field of the Invention

The present invention relates generally to the fields of oncology, pathology, immunology, molecular biology and gene therapy. More particularly, it concerns the use of p53 gene therapy to increase chemotherapy efficacy and stimulate anti-tumor immune responses in patients with tumors, such as breast cancer.

II. Description of Related Art

The occurrence of cancer is so high that over 500,000 deaths per year are attributed to cancer in the United States alone. Currently, there are few effective options for the treatment of many common cancer types. The course of treatment for a given individual depends on the diagnosis, the stage to which the disease has developed and factors such as age, sex and general health of the patient. The most conventional options of cancer treatment are surgery, radiation therapy and chemotherapy. There are limitations associated with each of these modalities, particular in the treatment of solid tumors. For example, local-regional recurrence of cancer remains a significant problem for some tumor types after surgical excision.

Radiation therapy may be accompanied by side effects, including skin irritation, difficulty swallowing, dry mouth, nausea, diarrhea, hair loss and loss of energy (Curran, 1998; Brizel, 1998). Later side effects include fibrosis, loss of skin blood vessels, intestinal damage, and bowel obstruction. Organ failure, such as the loss of kidney or heart function, may also occur. Radiation to the brain can cause delayed mental problems, including memory loss.

Regarding chemotherapy, its efficacy is often is limited by the difficulty of achieving drug delivery throughout solid tumors (el-Kareh and Secomb, 1997). Another major side effect of chemotherapeutic agents is that they can affect normal tissue cells, with the cells most likely to be affected being those that divide rapidly (e.g., bone marrow, gastrointestinal tract, reproductive system and hair follicles). Other toxic side effects of chemotherapy drugs are sores in the mouth, difficulty swallowing, dry mouth, nausea, diarrhea, vomiting, fatigue, bleeding, hair loss and infection.

It is now well established that a variety of cancers are caused, at least in part, by genetic abnormalities that result in either the overexpression of cancer causing genes, called “oncogenes,” or from loss of function mutations in protective genes, often called “tumor suppressor” genes. An important gene of the latter category is p53-a 53 kD nuclear phosphoprotein that controls cell proliferation. Mutations to the p53 gene and allele loss on chromosome 17p, where this gene is located, are among the most frequent alterations identified in human malignancies. The p53 protein is highly conserved through evolution and is expressed in most normal tissues. Wild-type p53 has been shown to be involved in control of the cell cycle (Mercer, 1992), transcriptional regulation (Fields and Jang, 1990; Mietz et al., 1992), DNA replication (Wilcock and Lane, 1991; Bargonetti et al., 1991), and induction of apoptosis (Yonish-Rouach et al., 1991; Shaw et al., 1992).

Various mutant p53 alleles are known in which a single base substitution results in the synthesis of proteins that have quite different growth regulatory properties and, ultimately, lead to malignancies (Hollstein et al., 1991). In fact, the p53 gene has been found to be the most frequently mutated gene in common human cancers (Hollstein et al., 1991; Weinberg, 1991), and is particularly associated with those cancers linked to cigarette smoke (Hollstein et al., 1991; Zakut-Houri et al., 1985).

The overexpression of p53 in breast tumors has also been documented (Casey et al., 1991). Interestingly, however, the beneficial effects of p53 are not limited to cancers that contain mutated p53 molecules. In a series of papers, Clayman et al. (1994; 1995a; 1995b) demonstrated that growth of cancer cells expressing wild-type p53 molecules was nonetheless inhibited by expression of p53 from a viral vector.

As a result of these findings, considerable effort has been placed into p53 gene replacement therapy. Retroviral delivery of p53 to humans was reported by Roth et al. (1996), who used a retroviral vector containing the wild-type p53 gene under control of a beta-actin promoter to mediate transfer of wild-type p53 into 9 human patients with non-small cell lung cancers by direct injection. Tumor regression was noted in three patients, and tumor growth stabilized in three other patients. Similar studies have been conducted using adenovirus to deliver p53 to human patients with squamous cell carcinoma of the head and neck (SCCHN) (Clayman et al., 1998). Surgical and gene transfer-related morbidities were minimal, and the overall results provided preliminary support for the use of Ad-p53 gene transfer as a surgical adjuvant in patients with advanced SCCHN.

Immunotherapy, a rapidly evolving area in cancer research, is yet another option for the treatment of certain types of cancer. In general, immunotherapy involves the stimulation of a humoral immune response to tumor or cancer cell antigens, or the stimulation of a cellular immune response to the cancer. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

An alternative aspect of immunotherapy is to enhance anticancer effects with immune stimulatory effects. Examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons), and (IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Combining immune stimulating molecules, either as proteins or using gene delivery in combination with a tumor suppressor such as mda-7 has been shown to enhance anti-tumor effects (Ju et al., 2000).

Various studies support the idea that tumor infiltration by lymphocytes is associated with an anti-tumor immune response (Hadden, 1999; Topalian et al., 1989). This has been shown, for example, by isolating tumor infiltrating lymphocytes from melanoma tissue and culturing the cells under conditions that allow for expansion of the lymphocyte population. When infused along with the cytokine IL-2 into patients with melanoma, the expanded lymphocytes are capable of targeting and re-infiltrating the melanoma tumors, with positive effects in many of the patients (Rosenberg, 2001).

There have been few studies exploring the use of p53 in immunotherapy. For example, in an in vitro assay, p53 mutant peptides capable of binding to HLA-A2.1 and inducing primary cytotoxic T lymphocyte (CTL) responses were identified (Houbiers et al., 1993). In a study in which synthetic p53 mutant and wild-type peptides were screened for immunogenicity in mice, it was observed that only mutant p53 epitopes were capable of eliciting a CTL response (Bertholet et al., 1997). In contrast, the immunization of BALB/c mice with bone marrow-derived dendritic cells (DC) in the presence of GM-CSF/IL-4 and prepulsed with the H-2 Kd binding wild-type p53 peptide (232-240) was observed to induce p53 anti-peptide CTL response (Ciernik et al., 1996; Gabrilovich et al., 1996; Yanuck et al., 1993; DeLeo, 1998; Mayordomo et al., 1996).

Another effort at immunotherapy using p53 involves the intradermal and intramuscular injection of naked plasmic DNA encoding human wild-type p53 and the intravenous injection of human wild-type p53 presented by a recombinant canarypox vector (Hurpin et al., 1998). It was not shown whether this method was of benefit in the treatment of solid tumors. Recently, it has been proposed to induce an immune response in a subject with a tumor by intradermally administering dendritic cells that have been transduced with p53 (U.S. Patent App. Pub. No. 20030045499).

Unfortunately, the immune response generated with immunotherapy regimens is often not sufficient to prevent most tumors. This is a particular problem for relatively large solid tumors with rapidly dividing cells. Thus, there is the need for improved methods of augmenting the immune response such that growth of abnormal cells can be halted to facilitate tumor destruction by immune effector cells. Such methods can be applied as novel forms of cancer therapy, either alone or in combinations with other standard forms of cancer therapy.

### SUMMARY OF THE INVENTION

The present inventors have discovered that administration of a p53 expression construct to a tumor in an immunocompetent subject results in significant tumor regression as a result of generation of a previously undescribed immunological mechanism. In one clinical protocol that was an open label, non-randomized Phase II study, for example, the inventors administered to a cohort of subjects with locally advanced breast cancer a treatment regimen employing a recombinant adenovirus expressing p53 and two chemotherapeutic agents, docetaxel and doxorubicin. The recombinant adenovirus expressing p53 was administered by intratumoral injection, and the doxorubicin and docetaxel were administered intravenously on a 21-day cycle, with up to six cycles of treatment. Additional details regarding the clinical protocol are set forth in Example 1 below. Median tumor size decreased substantially from 8.00 cm at enrollment to 1.78 cm at the conclusion of the study.

Biopsy specimens of tumors showed extensive T-lymphocyte infiltrates in all specimens. Clinical response was demonstrated in 100% of the patients, with a majority demonstrating minimal pathological breast residual disease. Further, the treatment was found to be safe and well-tolerated. Moreover, activation of mature T-cells was associated with a lower residual disease, indicating a therapeutic role for these cells. These results indicate that administration of a p53 expression construct to a tumor can be an effective means of achieving significant tumor regression by promoting T-lymphocyte infiltration into the tumor. This invention thus represents a novel form of cancer therapy, which can be used either alone or in combination with more convention forms of cancer therapy.

The present invention generally pertains to novel methods for inducing an immune response in a tumor in an immunocompetent subject, comprising injecting a first expression construct comprising a nucleic acid segment encoding p53 into the tumor in an amount effective to induce an immune response in the tumor. The “immune response” is defined herein to refer to a response whereby the immune system of the subject recognizes a cell of the tumor as foreign. For example, in some embodiments, the immune response involves an infiltration of one or more T cells into the tumor. The T cells, for example, may be cytotoxic T cells. In other embodiments, the immune response involves an infiltration of one or more B cells into the tumor. Induction of an immune response may also involve induction of immunomodulators, such as cytokines. The mechanism of induction of an immune response is discussed in greater detail in the specification below.

A “tumor,” as discussed in greater detail below, refers to an abnormal growth of tissue resulting from an abnormal growth or multiplication of cells. Tumor, as used herein, also refers to a solid mass of tissue that is of sufficient size such that an immune response can be detected in the tissue. In certain particular embodiments, the tumor is a cancer, such as brain cancer, head & neck cancer, lung cancer, breast cancer, cervical cancer, bladder cancer, skin cancer, or rectal cancer. In a particular embodiment, the tumor is a breast cancer.

The subject can be any subject, such as a laboratory animal or a human, so long as the subject is immunocompetent. An “immunocompetent subject” is defined herein to refer to a subject who has the normal bodily capacity to develop an immune response following exposure to an antigen. There are numerous ways in which one could identify a subject. In certain particular embodiments, the subject is a patient with a tumor that is cancerous. The subject may or may not be a candidate for other treatment modalities. For example, in some embodiments, the subject is a patient with unresectable breast cancer. Reduction of tumor size in response to the therapeutic methods of the present invention may result in the patient being eligible for surgical resection or other therapeutic interventions.

Expression constructs encoding p53 are discussed in detail in the specification below. Both wild-type and mutant versions of p53 sequences are contemplated for the methods set forth herein.

The expression construct can be formulated in any manner known to those of ordinary skill in the art. For example, as set forth below, the expression construct can be formulated in a composition that includes one or more pharmaceutically effective carriers, or one or more additional therapeutic agents that can be applied in the treatment of a tumor. Formulations are addressed in greater detail in the specification below.

In some embodiments of the present methods, the first expression construct is injected more than one time into the tumor. For example, the second injection may be performed as repeat therapy after detecting an immune response in the tumor following the first injection.

In some embodiments, a second expression construct comprising a nucleic acid segment encoding p53 into the tumor is injected into the tumor concurrently or following injection of the first expression construct into the tumor. The first and second expression constructs are different. For example, the first and second expression construct may include different promoters. In some embodiments, the second expression construct may be injected after detecting an immune response in the tumor following injection of the first expression construct.

Injection of the expression construct comprising a nucleic acid encoding p53 can be by method or technique known to those of ordinary skill in the art. For example, injection can be intratumoral injection, wherein the expression construct is injected into the tumor tissue. Injection may also include injection to the perimeter of the tumor, such as to normal tissue encircling a tumor. Injection into tumor vasculature can also be performed. Methods of administration are discussed further in the specification below.

In certain particular embodiments, the expression construct is a viral expression construct. For example, the viral expression construct may be a retroviral construct, a herpesviral construct, an adenoviral construct, an adeno-associated viral construct, or a vaccinia viral construct. In certain particular embodiments, the viral expression construct is a replication-competent virus. In other embodiments, the viral expression construct is a replication-defective virus. Examples of viral expression constructs and detail regarding engineering of such constructs are addressed in greater detail below.

In further embodiments, the expression construct is comprised in a nonviral vector. For example, the nonviral vector may include a lipid. The lipid can be any lipid or mixture of lipids known to those of ordinary skill in the art. In certain embodiments, the vehicle is a DOTAP:cholesterol nanoparticles. Nanoparticles and lipid vehicles are addressed in detail in the specification below.

In certain embodiments, the nucleic acid segment encoding p53 is under the control of a promoter that is active in cells of the subject. In some embodiments, the promoter is active in the tumor cells. Examples of such promoters are detailed below in the specification, and include CMV IE, RSV LTR, β-actin, Ad-E1, Ad-E2 or Ad-MLP.

In certain embodiments of the present invention, the methods further include detecting the stimulated immune response against the tumor. Any method known to those of ordinary skill in the art can be used to detect the stimulated immune response against the tumor. Exemplary methods are detailed in the specification as follows. In certain particular embodiments, the immune response is detected by detecting tumor swelling, such as by palpation or by imaging studies, within about one month following injection. Exemplary imaging studies that can be applied for this purpose include CT, MRI, ultrasound, and PET.

In other embodiments, histological analysis is performed on a tumor biopsy specimen or a surgical specimen following excision. Thus, for example, detecting an immune response may include detecting T-cells in the tumor, measuring T cell specific proteins in the tumor, and/or measuring T cell specific nucleic acids in the tumor. In certain particular embodiments, the stimulated immune response is detected histologically by evaluating T-cell lymphocyte infiltration into the tumor.

The expression constructs comprising a nucleic acid segment encoding p53 can be administered one time, or more than one time. A therapeutically effective amount of the expression construct is an amount, or dosage, that is known or suspected to reduce the number of tumor cells; reduce the tumor size; inhibit (i.e., slow to some extent and preferably stop) tumor cell infiltration into peripheral organs; inhibit (i.e., slow to some extent and preferably stop) tumor metastasis; inhibit, to some extent, tumor growth; and/or relieve to some extent one or more of the symptoms associated with the disorder.

In certain particular embodiments of the present invention, the administration of the expression construct encoding p53 further includes administration of one or more chemotherapeutic agents to the subject. A list of exemplary chemotherapeutic agents is set forth in the specification below. In certain particular embodiments, the subject is treated with one or more chemotherapeutic agents selected from the group consisting of cisplatinum, cyclophosphamide, 5-FU, gemcitabine, methotrexate, doxorubicin, docetaxel, paclitaxel, vinorelbine, and camptothecin. The chemotherapeutic agents may be administered concurrently, prior to, or consecutively with the therapeutic expression construct. If administered concurrently, the chemotherapeutic agent may be formulated in a single composition with the expression construct, or formulated for separate administration.

In certain embodiments of the present invention, the method is further defined as a method of sensitizing the tumor of the subject to chemotherapy. The dosage of chemotherapeutic agent may be a standard dose that is given in existing protocols, or a lower dose in view of the immunostimulation associated with administration of the p53 expression construct.

The first and second expression constructs may be administered a single time, or more than one time, as set forth above. Further, any of the methods set forth herein can be combined with one or more other forms of antitumor therapy, such as surgical therapy, gene therapy, other forms of immunotherapy, radiation therapy, or chemotherapy.

In certain embodiments of the present invention, the methods of the present invention involve identifying a subject. There are numerous ways in which one could identify a subject. Examples include interview, questionnaires, physical examination, and referral. For example, physical examination of a group of patients may be used to identify patients with tumors of the breast. One of ordinary skill in the art would be familiar with methods of identifying a subject.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a diagram of the treatment plan for the study;

FIG. 2 is a graph showing the reduction in size of primary lesions of various patients, post treatment;

FIG. 3 is a graph showing the reduction in size of axillary node lesions of various patients, post treatment;

FIG. 4 is a graph showing that administration of Advexin® correlates with detectable p53 mRNA expression;

FIG. 5 shows representative tissue sections of tumor biopsy samples; and

FIG. 6 provides representative tissue sections of immunostained T-cells.

### DESCRIPTION OF THE ILLUSTRATIVE EMBODIMENTS

The present invention is based on the inventors' discovery that administration of a p53 expression construct to a tumor of an immunocompetent individual is sufficient to effectively promote significant tumor regression by means of a previously undescribed immunological mechanism. In particular, administration to of the p53 expression construct to a tumor has been found to be associated with an extensive T-lymphocyte infiltrate within the tumor. The activation of mature T-cells was found to be associated with a lower residual disease, indicating a therapeutic role for these T-cells.

The present invention has advantages over the prior art. In particular, unlike intradermal administration of the construct, the present methods have been shown to result in a substantial infiltration of lymphocytes in the tumor with associated tumor regression. Further, the immune response is generated locally within the tumor, and would be expected to result in fewer side effects. Further, the immune response is generated without the need to obtain dendritic cells from the subject, transduce the dendritic cells, and administer them to the subject. Again, the immune response is generated where it is needed—within the tumor itself. The direct introduction of the p53 expression construct into the tumor may result in a more substantial T-cell infiltration compared to other methods of immunotherapy using p53, which may account for the greater therapeutic efficacy of the present methods.

A. Nucleic Acid Segments Encoding p53

Certain embodiments of the present invention concern nucleic acid segments encoding p53. In certain aspects, both wild-type and mutant versions of these sequences will be employed. The term “nucleic acid” is well known in the art. A “nucleic acid segment” as used herein, will generally refer to a molecule (i.e., a strand) of DNA, RNA or a derivative or analog thereof, comprising a nucleotide base. A nucleotide base includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide.” The term “oligonucleotide” refers to a molecule of between about 8 and about 100 nucleotide bases in length. The term “polynucleotide” refers to at least one molecule of greater than about 100 nucleotide bases in length.

B. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. Various mechanisms of oligonucleotide synthesis may be used, such as those methods disclosed in, U.S. Pat. Nos. 4,659,774; 4,816,571; 5,141,813; 5,264,566; 4,959,463; 5,428,148; 5,554,744; 5,574,146; 5,602,244, each of which are incorporated herein by reference.

A non-limiting example of an enzymatically produced nucleic acid include nucleic acids produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

C. Expression Constructs

In accordance with the present invention, it will be desirable to produce p53 proteins in a cell. Expression typically requires that appropriate signals be provided in the vectors or expression cassettes, and which include various regulatory elements, such as enhancers/promoters from viral and/or mammalian sources that drive expression of the genes of interest in host cells. Elements designed to optimize messenger RNA stability and translatability in host cells may also be included. Drug selection markers may be incorporated for establishing permanent, stable cell clones.

Viral vectors are selected eukaryotic expression systems. Included are adenoviruses, adeno-associated viruses, retroviruses, herpesviruses, lentivirus and poxviruses including vaccinia viruses and papilloma viruses including SV40. Viral vectors may be replication-defective, conditionally-defective or replication-competent. Also contemplated are non-viral delivery systems, including lipid-based vehicles.

1. Vectors and Expression Constructs

The term “vector” is used to refer to a carrier nucleic acid molecule into which a nucleic acid sequence can be inserted for introduction into a cell where it can be replicated and/or expressed. A nucleic acid sequence can be “exogenous” or “heterologous” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1996, both incorporated herein by reference).

The terms “expression vector” and “expression construct” refer to any type of genetic construct comprising a nucleic acid coding for a RNA capable of being transcribed. In some cases, RNA molecules are then translated into a protein, polypeptide, or peptide. Expression constructs can contain a variety of “control sequences,” which refer to nucleic acid sequences necessary for the transcription and possibly translation of an operable linked coding sequence in a particular host cell. In addition to control sequences that govern transcription and translation, vectors and expression constructs may contain nucleic acid sequences that serve other functions as well, as described below.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of an RNA into a gene product. In other embodiments, expression only includes transcription of the nucleic acid.

2. Promoters

A “promoter sequence” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence. Together, an appropriate promoter or promoter/enhancer combination, and a gene of interest, comprise an expression construct. One or more expression constructs may be present in a given nucleic acid vector or expression vector.

A promoter may be one naturally associated with a gene or sequence, as may be obtained by isolating a portion the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. In certain aspect of the invention a heterologous promoter may be a chimeric promoter, where elements of two or more endogenous, heterologous or synthetic promoter seqeunces are operatively coupled to produce a recombinant promoter.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Such promoters may be used to drive reporter expression, e.g., β-galactosidase or luciferase to name a few. Furthermore, it is contemplated the control sequences that direct transcription and/or expression of sequences within non-nuclear organelles such as mitochondria, chloroplasts, and the like, can be employed as well.

A promoter and/or enhancer will typically be used that effectively directs the expression of the DNA segment in a cell type, organelle, and organism chosen for expression. Those of skill in the art of molecular biology generally know the use of promoters, enhancers, and cell type combinations for protein expression, for example, see Sambrook et al., (1989), incorporated herein by reference. The promoters employed may be constitutive, tissue-specific, inducible, and/or useful under the appropriate conditions to direct expression of the introduced DNA segment, such as is advantageous in the production of recombinant proteins and/or peptides. The promoter may be heterologous or endogenous or a combination thereof.

A promoter may be functional in a variety of tissue types and in several different species of organisms, or its function may be restricted to a particular species and/or a particular tissue or cell type. Further, a promoter may be constitutively active, or it may be selectively activated by certain substances (e.g., a tissue-specific factor), under certain conditions (e.g., hypoxia, or the presence of an enhancer element in the expression cassette containing the promoter), or during certain developmental stages of the organism (e.g., active in fetus, silent in adult).

In certain embodiments of the present invention, the promoter is active in a tumor cell. “Tumor” is defined elsewhere in this specification. The tumor cell can be a hyperplastic cell, or it can be a normal cell within a tumor, such as a vascular endothelial cell. A promoter that is active in a tumor cell is a promoter capable of driving transcription of a gene in a tumor cell while remaining largely “silent” or expressed at low relatively low levels in other cells. It will be understood, however, that such promoters may have a detectable amount of “background” or “base” activity in those tissues where they are silent. The degree to which a promoter is selectively activated in a target tissue can be expressed as a selectivity ratio (activity in a target tissue/activity in a control tissue). In this regard, a promoter useful in the practice of the present invention typically has a selectivity ratio of greater than about 2, 3, 4, or 5. Preferably, the selectivity ratio is greater than about 10 or 15.

The level of expression of a gene under the control of a particular promoter can be modulated by manipulating the promoter region. For example, different domains within a promoter region may possess different gene-regulatory activities. The roles of these different regions are typically assessed using vector constructs having different variants of the promoter with specific regions deleted (i.e., deletion analysis). Vectors used for such experiments typically contains a reporter sequence, which is used to determine the activity of each promoter variant under different conditions. Application of such a deletion analysis enables the identification of promoter sequences containing desirable activities and thus identifying a particular promoter domain, including core promoter elements.

Examples of promoters particularly active in tumors include, for example, an hTERT promoter sequence, a CEA promoter sequence, a PSA promoter sequence, a probasin promoter sequence, a ARR2PB promoter sequence, an AFP promoter sequence a human alpha-lactalbumin promoter sequence, an ovine beta-lactoglobulin promoter sequence, a U6 promoter sequence, an H1 promoter sequence, a 7SL promoter sequence, a human Y promoter sequence, a human MRP-7-2 promoter sequence, an adenovirus VA1 promoter sequence, a human tRNA promoter sequence, a 5S ribosomal RNA promoter sequence, or a functional hybrid or a combination of any of these promoter sequences. Other examples include hypoxia-specific promoter sequences, such as a hypoxic response element (HRE) or a hypoxia inducible factor. Examples of hypoxia inducible factors include HIF-1 alpha, HIF-2alpha, or HIF-3alpha.

One of ordinary skill in the art would be familiar with other promoter sequences that can be included in the context of the present invention. Examples of these promoters are included in Table 1.

TABLE 1
PROMOTER AND/OR ENHANCER

| Promoter/Enhancer | References |
|---|---|
| Immunoglobulin Heavy Chain | Banerji et al., 1983; Gilles et al., 1983; Grosschedl et |
| | al., 1985; Atchinson et al., 1986, 1987; Imler et al., |
| | 1987; Weinberger et al., 1984; Kiledjian et al., 1988; |
| | Porton et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick et al., 1989; Johnson et |
| (MCK) | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al, 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et al., |
| | 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency | Muesing et al., 1987; Hauber et al., 1988; Jakobovits |
| Virus | et al., 1988; Feng et al., 1988; Takebe et al., 1988; |
| | Rosen et al., 1988; Berkhout et al., 1989; Laspia et al., |
| | 1989; Sharp et al., 1989; Braddock et al., 1989 |
| Cytomegalovirus (CMV) | Weber et al., 1984; Boshart et al., 1985; Foecking et |
| | al., 1986 |
| Gibbon Ape Leukemia Virus | Holbrook et al., 1987; Quinn et al., 1989 |

3. Selectable Markers

In certain embodiments of the invention, a nucleic acid construct of the present invention may be identified by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Examples of selectable and screenable markers are well known to one of skill in the art.

4. Reporters

The term “reporter,” “reporter gene” or “reporter sequence” as used herein refers to any genetic sequence or encoded polypeptide sequence that is detectable and distinguishable from other genetic sequences or encoded polypeptides present in cells. In certain embodiments of the present invention, the expression construct includes such a reporter sequence. Preferably, the reporter sequence encodes a protein that is readily detectable either by its presence, or by its activity that results in the generation of a detectable signal. In certain aspects, a detectable moiety may include a fluorophore, a luminophore, a microsphere, an enzyme, a polypeptide, a polynucleotide, and/or a nanosphere, all of which may be coupled to an antibody or a ligand that recognizes and/or interacts with a reporter.

In various embodiments, a nucleic acid sequence of the invention comprises a reporter nucleic acid sequence or encodes a product that gives rise to a detectable polypeptide. A reporter is or encodes a reporter molecule which is capable of directly or indirectly generating a detectable signal. Generally, although not necessarily, the reporter gene encodes RNA and/or detectable protein that are not otherwise produced by the cells. Many reporter genes have been described, and some are commercially available for the study of gene regulation. See, for example, Alam and Cook (1990), the disclosure of which is incorporated herein by reference. Signals that may be detected include, but are not limited to color, fluorescence, luminescence, isotopic or radioisotopic signals, cell surface tags, cell viability, relief of a cell nutritional requirement, cell growth and drug resistance. Reporter sequences include, but are not limted to, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, G-protein coupled receptors, somatostatin receptors, CD2, CD4, CD8, the influenza hemagglutinin protein, symporters (such as NIS) and others well known in the art, to which high affinity antibodies or ligands directed thereto exist or can be produced by conventional means, and fusion proteins comprising a membrane bound protein appropriately fused to an antigen tag domain from, among others, hemagglutinin or Myc.

5. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the 6

6. Polyadenylation Signals

One may include a polyadenylation signal in the expression construct to effect proper polyadenylation of the transcript. The nature of the polyadenylation signal is not believed to be crucial to the successful practice of the invention, and/or any such sequence may be employed. Specific embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

7. Termination Signals

The vectors or constructs of the present invention may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. One of ordinary skill in the art would be familiar with termination signals.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

8. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

9. IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781).

D. Viral Vectors

“Viral vectors,” or “viral expression constructs,” are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encodes one or more candidate substance or other components such as, for example, an immunomodulator or adjuvant for the candidate substance. Non-limiting examples of virus vectors that may be used to deliver a nucleic acid of the present invention are described below.

1. Adenoviral Vectors

a. Virus Characteristics

In certain embodiments of the present invention, the viral expression construct is an adenoviral construct. Adenovirus is a non-enveloped double-stranded DNA virus. The virion consists of a DNA-protein core within a protein capsid. Virions bind to a specific cellular receptor, are endocytosed, and the genome is extruded from endosomes and transported to the nucleus. The genome is about 36 kB, encoding about 36 genes. In the nucleus, the “immediate early” E1A proteins are expressed initially, and these proteins induce expression of the “delayed early” proteins encoded by the E1B, E2, E3, and E4 transcription units. Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP).

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

Adenovirus may be any of the 51 different known serotypes or subgroups A-F. Adenovirus type 5 of subgroup C is the human adenovirus about which the most biochemical and genetic information is known, and it has historically been used for most constructions employing adenovirus as a vector. Recombinant adenovirus often is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Viruses used in gene therapy may be either replication-competent or replication-deficient. Generation and propagation of the adenovirus vectors which are replication-deficient depends on a helper cell line, the prototype being 293 cells, prepared by transforming human embryonic kidney cells with Ad5 DNA fragments; this cell line constitutively expresses E1 proteins (Graham et al., 1977). However, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Adenovirus growth and manipulation is known to those of skill in the art, and exhibits broad host range in vitro and in vivo. This group of viruses can be obtained in high titers, e.g., 10⁹-10¹³ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Engineering

As stated above, Ad vectors are based on recombinant Ad's that are either replication-defective or replication-competent. Typical replication-defective Ad vectors lack the E1A and E1B genes (collectively known as E1) and contain in their place an expression cassette consisting of a promoter and pre-mRNA processing signals, which drive expression of a foreign gene. These vectors are unable to replicate because they lack the E1A genes required to induce Ad gene expression and DNA replication. In addition, the E3 genes can be deleted because they are not essential for virus replication in cultured cells. It is recognized in the art that replication-defective Ad vectors have several characteristics that make them suboptimal for use in therapy. For example, production of replication-defective vectors requires that they be grown on a complementing cell line that provides the E1A proteins in trans.

Several groups have also proposed using replication-competent Ad vectors for therapeutic use. Replication-competent vectors retain Ad genes essential for replication, and thus do not require complementing cell lines to replicate. Replication-competent Ad vectors lyse cells as a natural part of the life cycle of the vector. An advantage of replication-competent Ad vectors occurs when the vector is engineered to encode and express a foreign protein. Such vectors would be expected to greatly amplify synthesis of the encoded protein in vivo as the vector replicates. For use as anti-cancer agents, replication-competent viral vectors would theoretically be advantageous in that they would replicate and spread throughout the tumor, not just in the initially infected cells as is the case with replication-defective vectors.

Yet another approach is to create viruses that are conditionally-replication competent. Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178). This phenotype is reportedly accomplished by using recombinant adenoviruses containing a mutation in the E1B region that renders the encoded E1B-55K protein incapable of binding to p53 and/or a mutation(s) in the E1A region which make the encoded E1A protein (p289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB.

Another replication-competent adenovirus vector has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

The present invention has taken advantage of the differential expression of telomerase in dividing cells to create novel adenovirus vectors which overexpress an adenovirus death protein and which are replication-competent in and, preferably, replication-restricted to cells expressing telomerase. Specific embodiments include disrupting E1A's ability to bind p300 and/or members of the Rb family members. Others include Ad vectors lacking expression of at least one E3 protein selected from the group consisting of 6.7K, gp19K, RIDα (also known as 10.4K); RIDβ (also known as 14.5K) and 14.7K. Because wild-type E3 proteins inhibit immune-mediated inflammation and/or apoptosis of Ad-infected cells, a recombinant adenovirus lacking one or more of these E3 proteins may stimulate infiltration of inflammatory and immune cells into a tumor treated with the adenovirus and that this host immune response will aid in destruction of the tumor as well as tumors that have metastasized. A mutation in the E3 region would impair its wild-type function, making the viral-infected cell susceptible to attack by the host's immune system. These viruses are described in detail in U.S. Pat. No. 6,627,190.

Other adenoviral vectors are described in U.S. Pat. Nos. 5,670,488; 5,747,869; 5,932,210; 5,981,225; 6,069,134; 6,136,594; 6,143,290; 6,210,939; 6,296,845; 6,410,010; and 6,511,184; U.S. Publication No. 2002/0028785; U.S. Publication No. 2004/0213764, and U.S. patent application Ser. No. 09/351,778, each of which is specifically incorporated by reference in its entirety.

2. AAV Vectors

The nucleic acid may be introduced into the cell using adenovirus-assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the methods of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as therapeutic vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene, which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

E. Non-Viral Delivery

In certain embodiments of the present invention, the expression construct is comprised in a nonviral vector. This means that the expression construct is comprised within a delivery agent other than a viral vector. A “delivery agent” is defined herein to refer to any agent or substance, other than a viral vector, that facilitates the delivery of the nucleic acid to a target cell of interest. Exemplary delivery agents include lipids and lipid formulations, including liposomes. In certain embodiments, the lipid is comprised in nanoparticles. A “nanoparticle” is defined herein to refer to a submicron particle. For example, the nanoparticle may have a diameter of from about 1 to about 100 nanometers.

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (WO 98/07408). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these liposomes include colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

The liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

A nucleic acid for nonviral delivery may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

F. Detecting Stimulated Immune Response Against a Tumor

Certain embodiments of the present invention include assessing a stimulated immune response against a tumor following administration to the tumor of an expression construct comprising a nucleic acid encoding p53. The nature and extent of an anti-tumor immune response can be assessed by one or more methods known to those of ordinary skill in the art.

1. Measuring Tumor Size

It is suspected that infiltration of lymphocytes into a tumor tissue, which is one form of immune response, may involve an increase in tumor size. Thus, measuring or obtaining an indication of tumor size relative to pretreatment size is one form of detecting a stimulated immune response in a tumor. For example, this can be accomplished by palpation of the tumor by the physician to detect tumor swelling or enlargement. Alternatively, tumor size can be detected by measuring the size of the tumor using imaging technology. Any method of imaging known to those of ordinary skill in the art can be used. For example, imaging may via a CAT scan, MRI, ultrasound, PET scanning, and so forth.

2. Evaluation of Immune Response at the Cellular Level

Anti-tumor immune responses at the cellular level may also be assessed by histological examination of tumor tissue obtained by biopsy or by surgical excision of the tumor following injection of the therapeutic expression construct.

Tumor infiltrating lymphocyte populations may include both CD4⁺ and CD8⁺ T cells. As is known, CD4 and CD8 are membrane proteins associated with the T cell receptor, and are important in the recognition of antigen by T cells. CD4⁺ T cells recognize antigen in the context of major histocompatibility complex (MHC) Class II proteins, while CD8⁺ T cells recognize antigen in the context of MHC Class I proteins. Both types of T cells contain CD3, a complex of 5 polypeptides associated with the T cell receptor. Because of this association, CD3 can be used as a general marker for T cells. T cells can be characterized by identifying CD3, CD4 or CD8 antigens. In contrast, B-cells can be identified by the presence of CD20, an antigen expressed in most B-cells but not in T-cells.

In some embodiments, the immune response is an immune response associated with an increase in number of T cells into the tumor. Thus, assessment of the immune response may include immunohistochemical analysis of T-cell populations infiltrating tumor tissue, by molecular analysis of T cell proteins or nucleic acids present in the tumor, or by other methods well known in the art.

The extent of T-cell infiltration into a tumor can be evaluated by immunodetection methods. For example, infiltrating T-cells can be detected in formalin-fixed, paraffin-embedded tumor tissue sections by immunostaining cells with an antibody to CD3, CD4 or CD8. Immunodetection methods are well-known to those of ordinary skill in the art.

The immune response may also be an increase in B cell infiltration into the tumor. B cells can be detected with an antibody to CD20. The detection of immune complexes between antibody and antigen is well known in the art and may be achieved through the application of numerous approaches. These methods are generally based upon the detection of a label or marker, such as any of radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Additional advantages can be found through the use of a secondary binding ligand such as a second antibody and/or a biotin/avidin ligand binding arrangement, as is known in the art. The method of preparing tissue blocks from particulate specimens has been successfully used in previous studies, and/or is well known to those of skill in the art (Brown et al., 1990; Abbondanzo et al., 1990; Allred et al., 1990).

T-cell infiltration can also be evaluated by methods that detect proteins or nucleic acids specific to T-cells. For example, T-cell specific proteins such as CD3, CD4 and CD8 can be detected by enzyme linked immunosorbent assay (ELISA) or radioimmunoassay (RIA) of tumor biopsy protein preparations. Such assays, along with dot blotting, western blotting, and the like, are well known in the art. Alternatively, T-cell specific RNA molecules, such as RNAs for CD3, CD4 and CD8, can be detected by various methods including Northern blotting, RNA dot blotting, detection on DNA chips, and reverse transcription-polymerase chain reaction (RT-PCR) analysis.

As an example, for RT-PCR analysis of human CD3 RNA, total RNA can be extracted from a tumor biopsy sample. The RNA can be reverse transcribed into DNA, and the synthesized DNA can be amplified using the following human CD3 δ chain primers: GGTTCGGTACTTCTGACT (sense) (SEQ ID NO:1) and TGGTTTTGACTTGTTCTG (antisense) (SEQ ID NO:2). A sample can be amplified with Taq polymerase under the following conditions: 94° C. for 1 min, 48° C. for 1 min, 72° C. for 1 min, 32 cycles. Amplification products can be identified by electrophoresis through a 1.5% agarose gel followed by ethidium bromide staining (Airoldi et al., 2000).

G. Tumors

The present invention contemplates methods for treating a tumor in a subject. As used herein, a “tumor” refers to an abnormal growth of tissue resulting from an abnormal growth or multiplication of cells. Tumor, as used herein, also refers to a solid mass of tissue that is of sufficient size such that an immune response can be detected in the tissue. A tumor may be benign, premalignant, or malignant (i.e., cancerous). A tumor may be a primary tumor, or a metastatic lesion.

The terms “cancer” and “cancerous” refer to or describe the physiological condition in mammals that is typically characterized by unregulated cell growth. Examples of cancers that are associated with tumor formation include brain cancer, head & neck cancer, esophageal cancer, tracheal cancer, lung cancer, liver cancer stomach cancer, colon cancer, pancreatic cancer, breast cancer, cervical cancer, uterine cancer, bladder cancer, prostate cancer, testicular cancer, skin cancer, rectal cancer, and lymphomas. One of ordinary skill in the art would be familiar with the many disease entities that can be associated with tumor formation.

It is proposed that this approach will provide clinical benefit, defined broadly as any of the following: reducing primary tumor size, reducing occurrence or size of metastasis, reducing or stopping tumor growth, inhibiting tumor cell division, killing a tumor cell, inducing apoptosis in a tumor cell, reducing or eliminating tumor recurrence.

In certain embodiments of the present invention, the subjects are patients with unresectable tumors. Patients with unresectable tumors may be treated according to the present invention. As a consequence, the tumor may reduce in size, or the tumor vasculature may change such that the tumor becomes resectable. If so, standard surgical resection may be permitted.

H. Methods of Administration and Dosage

1. Methods of Administration

a. Definitions

The present invention generally pertains to methods of inducing an immune response in a tumor by injecting an expression construct encoding p53 into the tumor. Formulations of expression constructs encoding p53 are discussed in greater detail below.

In certain embodiments, a therapeutically effective amount of the expression construct is administered to the subject. The term “therapeutically effective amount” refers to an amount of a drug effective to treat a disease or disorder in a mammal. In the case of a tumor, a therapeutically effective amount of the expression construct may reduce the number of tumor cells; reduce the tumor size; inhibit (i.e., slow to some extent and preferably stop) tumor cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the expression construct may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

“Treatment” refers to both therapeutic treatment and prophylactic or preventative measures. Those in need of treatment include those already with the disorder as well as those in which the disorder is to be prevented.

b. Administration

The methods of the present invention pertain to injection of an expression construct encoding p53 into a tumor. Any method of injection into a tumor known to those of ordinary skill in the art is contemplated by the present invention. For example, the injection can be directly into the tumor tissue (i.e., intratumoral injection). Injection may also include injection to the perimeter of the tumor. Such injection to the perimeter of the tumor may or may not encircle the tumor. Alternatively, the injection can be directed into tumor vasculature. Administration regionally can include intravascular administration into one or more arteries that supply blood to a part of the body that includes the tumor. Thus, the injection can be local to the tumor, or regional to the tumor, or by any other method known to those of ordinary skill in the art.

Injection may also be directed to one or more sites of the body of the subject that are suspected of comprising a tumor, such as to lymph nodes in the region of a breast cancer.

In particular embodiments, one or more chemotherapeutic agents are administered concurrently or consecutively, such as part of a combination therapeutic regimen with the expression constructs encoding p53. This is discussed in greater detail in the specification below.

2. Dosage

An effective amount of the therapeutic or preventive agent is determined based on the intended goal, for example regression of a tumor.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

The quantity to be administered, both according to number of treatments and dose, depends on the subject to be treated, the state of the subject and the protection desired. Precise amounts of the therapeutic composition also depend on the judgment of the practitioner and are peculiar to each individual.

For example, in some embodiments of the present invention, the dose of viral vector ranges from 1×10¹¹ to 1×10¹⁵ viral particles for injection. In other embodiments, the dose of viral particles per injection is 1×10¹² to 1×10¹⁴. In certain particular embodiments, the dose of viral particles per injection is 1×10¹² to 5×10¹².

For various approaches, delayed release formulations could be used that provide limited but constant amounts of the therapeutic agent over an extended period of time.

I. Pharmaceutical Compositions

According to the present invention, an expression construct encoding p53 is injected into a tumor of a subject to induce an immune response for therapeutic purposes, such as for treatment of a cancerous tumor. Thus, in certain embodiments, the expression construct is formulated in a composition that is suitable for this purpose. The phrases “pharmaceutically” or “pharmacologically acceptable” refer to compositions that do not produce adverse, allergic, or other untoward reactions when administered to an animal or a human. As used herein, “pharmaceutically acceptable carrier” includes any and all solvents, carriers, dispersion media, coatings, antibacterial and antifungal agents, isotonic and absorption delaying agents and the like. The use of such media and agents for pharmaceutically active substances is well known in the art. Except insofar as any conventional media or agent is incompatible with the expression constructs of the present invention, its use in therapeutic compositions is contemplated. Supplementary active ingredients also can be incorporated into the compositions. For example, the supplementary active ingredient may be a chemotherapeutic agent, an additional immunotherapeutic agent, an additional expression construct encoding a therapeutic gene, and so forth.

Solutions of the active compounds can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present invention may include one or more pharmaceutically acceptable carriers. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. For parenteral administration in an aqueous solution, the solution should be suitably buffered if necessary and the liquid diluent first rendered isotonic with sufficient saline or glucose. These particular aqueous solutions are especially suitable for intravascular and intratumoral administration. In this connection, sterile aqueous media, which can be employed will be known to those of skill in the art in light of the present disclosure.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

J. Secondary Anti-Cancer Therapies and Combination Therapies

In certain embodiments of the present invention, the methods of the present invention pertain to treatment of a tumor in a subject, wherein the subject is undergoing secondary anticancer therapy.

A wide variety of cancer therapies, known to one of skill in the art, may be used in combination with the compositions of the claimed invention. Some of the existing cancer therapies and chemotherapeutic agents are described below. One of skill in the art will recognize the presence and development of other anticancer therapies which can be used in conjugation with the methods and compositions of the present invention, and will not be restricted to those forms of therapy set forth below.

In order to increase the effectiveness of an expression construct encoding a therapeutic agent, it may be desirable to combine these compositions with other agents effective in the treatment of tumors such as cancerous tumors. These compositions would be provided in a combined amount effective to kill or inhibit proliferation of the tumor cells. This process may involve contacting the tumor with the expression construct and the agent(s) or second factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent.

Alternatively, the p53 therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, p53 therapy is “A” and the secondary agent, such as radio- or chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described anti-tumor therapy.

In accordance with the present invention, additional therapies may be applied with further benefit to the patients. Such therapies include surgery, cytokines, toxins, drugs, dietary, or a non-p53-based gene therapy. Examples are discussed below.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

Additional information regarding chemotherapeutic agents can be found at on the world wide web at accessdata.fda.gov/scripts/cder/onctools/druglist.cfm, which is herein specifically incorporated by reference.

2. Subsequent Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

3. Additional Gene Therapy

a. Inducers of Cellular Proliferation

The proteins that induce cellular proliferation further fall into various categories dependent on function. The commonality of all of these proteins is their ability to regulate cellular proliferation. For example, a form of PDGF, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors Rb, p16, MDA-7, PTEN and C-CAM are specifically contemplated.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins, which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

d. p53 Gene Therapy

Human p53 gene therapy has been described in the literature since the mid-1990's. Any of the known methods of p53 gene therapy can be combined with the methods set forth herein to further augment the antitumor response. Roth et al. (1996) reported on retroviral-based therapy, Clayman et al. (1998) described adenoviral delivery. U.S. Pat. Nos. 6,017,524; 6,143,290; 6,410,010; and 6,511,847, and U.S. Patent Application No. 2002/0077313 each describe methods of treating patients with p53, and are hereby incorporated by reference. U.S. Pat. No. 5,747,469 and U.S. Application No. 2002/0006914 disclose p53 therapies in combination with radio- and chemotherapies, and are hereby incorporated by reference.

One particular mode of administration that can be used in conjunction with surgery is treatment of an operative tumor bed. Thus, in either the primary gene therapy treatment, or in a subsequent treatment, one may perfuse the resected tumor bed with the vector during surgery, and following surgery, optionally by inserting a catheter into the surgery site.

In another embodiment, the secondary treatment is a non-p53 gene therapy in which a second gene is administered to the subject. Delivery of a vector encoding p53 in conjunction with a second vector encoding one of the following gene products may be utilized. Alternatively, a single vector encoding both genes may be used. A variety of molecules are encompassed within this embodiment, some of which are described below.

4. Immunotherapy

Although the present invention involves p53 immunotherapy, additional immunotherapies can be employed in conjunction with p53 administration. For example, a p53 expression construct can be administered to a tumor along with a tumor antigen or a cytokine such as IL-2. Examples of non-p53 immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons, IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with Herceptin and chemotherapy has been shown to be more effective than the individual therapies.

5. Hormonal Therapy

The use of sex hormones is also contemplated in accordance with the methods described herein in the treatment of cancer. While the methods described herein are not limited to the treatment of a specific cancer, the use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

6. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Stereotactic radiotherapy is used to treat brain tumours. This technique directs the radiotherapy from many different angles so that the dose going to the tumour is very high and the dose affecting surrounding healthy tissue is very low. Before treatment, several scans are analysed by computers to ensure that the radiotherapy is precisely targeted, and the patient's head is held still in a specially made frame while receiving radiotherapy. Several doses are given.

Stereotactic radio-surgery (gamma knife) for brain tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy, the patient lies with their head in a large helmet, which has hundreds of holes in it to allow the radiotherapy beams through.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

7. Other Cancer Therapies

Examples of other cancer therapies include phototherapy, cryotherapy, toxin therapy, or hormonal therapy. One of skill in the art would know that this list is not exhaustive of the types of treatment modalities available for cancer and other hyperplastic lesions.

K Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Locally advanced breast cancer (LABC) is treated with induction chemotherapy (IC), surgery, radiotherapy +/− adjuvant hormonal therapy. Alterations in the p53 gene have been documented with higher frequency in LABC (50%-55%) compared gene with early breast cancer (25%-30%), and p53 mutations correlate with poor response to chemotherapy, more aggressive disease, early metastasis, and decreased survival rates. Although primary chemotherapy has been demonstrated to be effective in the management of LABC, other approaches are needed to improve response and survival in these patients.

A study was conducted to examine the therapeutic efficacy and safety of a treatment regimen employing a recombinant adenovirus (Advexin®) expressing p53 under the control of a CMV promoter, and two chemotherapeutic agents, docetaxel and doxorubicin. The study was conducted as an open label, non-randomized Phase II study in patients with LABC. The criteria for patient selection were: a) patients with Stage III A-B or localized Stage IV breast cancer with measurable disease; b) males and females at least 18 years old; c) Kamofsky≧70%; d) negative HIV test; and e) no prior chemotherapy for newly diagnosed breast cancer.

Patients were administered materials on a on 21-day cycle, with up to 6 cycles of treatment. On Day 1 of each cycle, Advexin® at a dose of 2.5×10¹² viral particles was administered by intratumoral injection; doxorubicin (50 mg/m2)+docetaxel (75 mg/m2) were administered by IV. On Day 2 of each cycle, patients were again administered Advexin® at a dose of 2.5×10¹² viral particles by intratumoral injection. Patients also received prophylactic G-CSF. Biopsies were taken at baseline and on Days 2,3, and 21 of Cycle 1 for evaluation of p53 mutation status and mRNA expression. Serum for analysis of Adenovirus Antibody (Ad5IgG) and p53 antibodies was taken at baseline and Day 21 of Cycle 1. Tumor assessment and assessment of loco regional lymph nodes were performed at baseline and cycles 4 and 6 (pre-surgery).

Mutations in the p53 gene of patients were determined by single strand conformational polymorphism (SSCP). The method of SSCP takes advantage of the observation that single-stranded nucleic acids can form secondary structures in solution under suitable conditions. The secondary structure depends on the base composition of the nucleic acid, which can be altered by a single nucleotide substitution. The alteration can result in a difference in electrophoretic mobility under nondenaturing conditions. The altered fragment can be detected by radioactive labeling or by silver staining.

The expression of p53 in biopsy samples was evaluated by RT-PCR. Tumor sections were analyzed for immune cells by immunostaining. Lymphocyte markers identified in assays included CD3, CD20, CD4, CD8 and CCR7 (another T-cell protein).

### EXAMPLE 2

### Results

Thirteen patients have been enrolled. The median age was 56 years (range 39-71). The clinical stage was determined to be: median tumor size 8.00 cm (range 5.00-11.00); III_{B-c}/III_{A}, 8 (75%)/4 (25%); T₄/T₃/T₂, 7(58%)/4(33%)/1(8%). Patients received up to 6 cycles of Advexin®/docetaxel/doxorubicin treatment. All patients (100%) had surgery.

Correlative studies on eleven patients were undertaken as follows:
a) p53 status: eight patients (73%) had p53 mutations;
b) presence of anti-p53 antibodies: four patients (37%) were positive at baseline for Anti p53 Abs (no change with treatment);
c) presence of anti-Ad5 antibodies: ten pts (90%) were positive for Ad5-Abs at baseline.

Safety analysis indicated that there were no Grade 3 side effects considered related to Advexin®. Eight patients (67%) had residual pathologic foci of disease in the breast of ≦10 mm. The mean size of the residual tumor in the breast was 1.78 cm. All specimens showed extensive T-lymphocytes infiltrate (CD20 20%, CD3, 80%; CD4 30% CD8 70%).

Median follow-up was 20 months (range 12-23+); Intent to-Treat (ITT) analysis indicated that three of thirteen patients (23%) had relapsed (12, 13 and 18 months) and 1 patient (8%) had died (13 months).

The results show that treatment with Advexin® in combination with docetaxel and doxorubicin was safe and well tolerated. Also, clinical response was achieved in 100% of the patients, with a majority demonstrating minimal pathological breast residual disease. Expression of p53 mRNA in treated breast lesions is detectable up to 19 days after treatment. Moreover, activation of mature T-cells was associated with a lower residual disease. This suggests a therapeutic role for these cells.

At a median follow up of 24.5 months, 77% of the patients were disease-free, and 93% were alive.

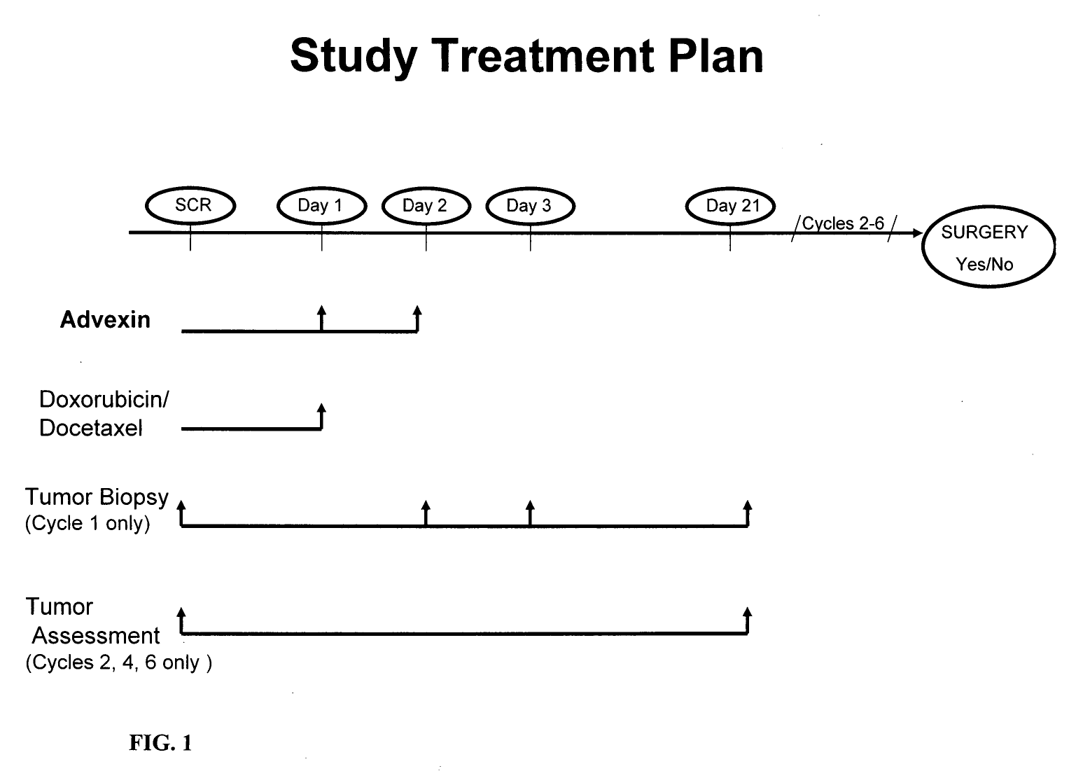

FIG. 1 is a diagram of the treatment plan for the study.

Currently, after 35 months of follow-up, 92 percent of the treated patients treated in the study are alive and 83 percent have survived without evidence of disease recurrence. Objective clinical responses were seen following the combined therapy in all of the patients with a median of 80 percent reduction in tumor size. Following tumor shrinkage, complete tumor removal by subsequent surgery was achieved in 100 percent of the patients. The results of the therapy with the addition of Advexin® are better than what would be expected from neoadjuvant chemotherapy treatment alone.

Thus, treatment with Advexin® results in activation of a local immune response at the site of the tumor was observed. Treated tumors were infiltrated with cells of the immune system that are known to participate in immune responses against tumors, which would be useful in controlling local disease as well as disease outside the breast. These data suggest that may be combined with neoadjuvant chemotherapy to improve patient outcomes by reducing tumor size thereby permitting complete surgical tumor removal. The dramatic reduction in tumor size allows use of less invasive surgeries that facilitate breast conservation.

The results of this study indicate that Advexin® may enhance the clinical benefit of chemotherapies without increasing their toxicity and support clinical applications of Advexin® in earlier phases of cancer treatment. Advexin® therapy may be combined with neoadjuvant chemotherapy to improve patient outcomes by reducing tumor size thereby permitting complete surgical tumor removal. The dramatic reduction in tumor size allows use of less invasive surgeries that facilitate breast conservation. The results of this study indicate that Advexin® may enhance the clinical benefit of chemotherapies without increasing their toxicity and support clinical applications of Advexin® in earlier phases of cancer treatment.

### EXAMPLE 3

### Patient Characteristics

Table 2 provides the baseline characteristics of the patients.

TABLE 2

| | |
|---|---|
| N | 13 |
| Median Age (range) | 56 yr (39-71) |
| Median Tumor Size | 8 cm (5-11) |
| N0/N1/N2/N3 | 1/3/4/4 |
| STAGE IIIA/IIIB/IIIC | 4/6/2 |
| p53 MUTATION | 8 pos/3 neg |

### EXAMPLE 4

### Adverse Events

Table 3 lists the adverse events considered possibly or probably related to Advexin® treatment in the Intent-To-Treat (ITT) patient population.

TABLE 3

| ADVERSE | | | |
|---|---|---|---|
| EVENT | MILD | MODERATE | SEVERE |
| SKIN | 2 | 0 | 0 |
| INFLAMMATION | | | |
| SKIN DISORDER | 1 | 0 | 0 |
| FEVER | 1 | 1 | 0 |
| FATIGUE | 1 | 0 | 0 |
| MYALGIA | 0 | 1 | 0 |
| BREAST PAIN | 0 | 1 | 0 |
| ANEMIA | 1 | 0 | 0 |
| WEIGHT LOSS | 1 | 0 | 0 |

### EXAMPLE 5

### Clinical Responses

Table 4 provides the clinical responses to Advexin® treatment.

TABLE 4

| | |
|---|---|
| Median Cycles Treated (range) | 6 (4-6) |
| Overall Response Rate: | |
| pCR | 0 |
| CR | 0 |
| PR | 12 (100%) |
| SD | 0 |
| Residual Disease: | |
| ≦1 cm | 8 (0.1-1 cm) |
| >1 cm | 4 (2.0-6.0 cm) |
| Median decrease in tumor size from | 79.4% (56.6-100) |
| baseline, target lesion (n = 12) | |
| Median decrease in tumor size from | 69.4% (38.6-100) |
| baseline, axillary nodes (n = 9) | |
| Median follow up | 34.5 mo |
| Median disease free interval | >32 mo |
| % disease free at 25 mo FU | 83% |
| Overall Survival at 25 mo FU | 92% |

### EXAMPLE 6

### Post Treatment Results and P53 mRNA Levels

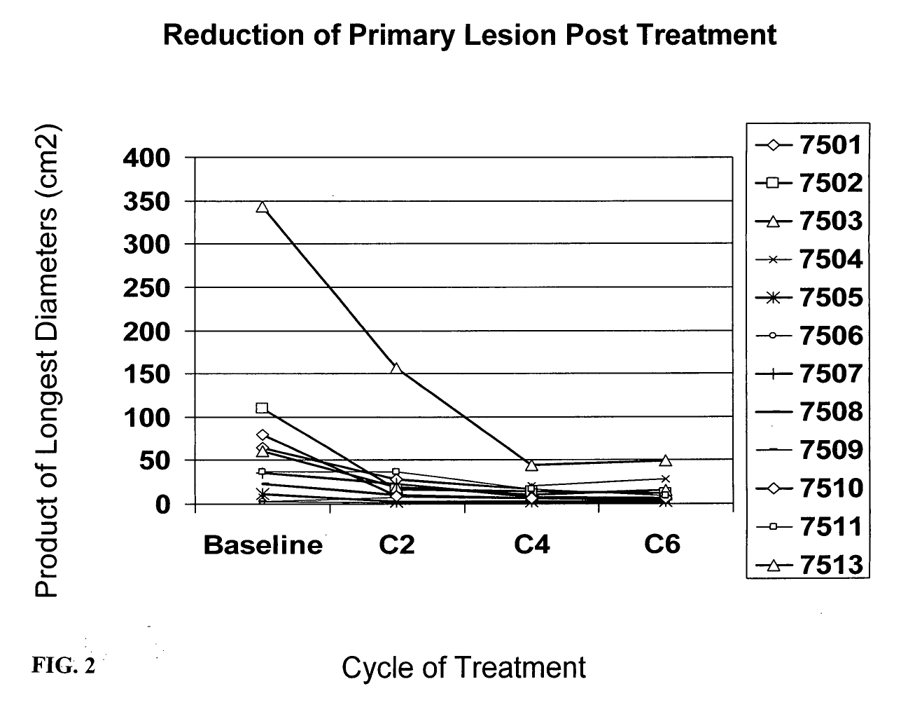

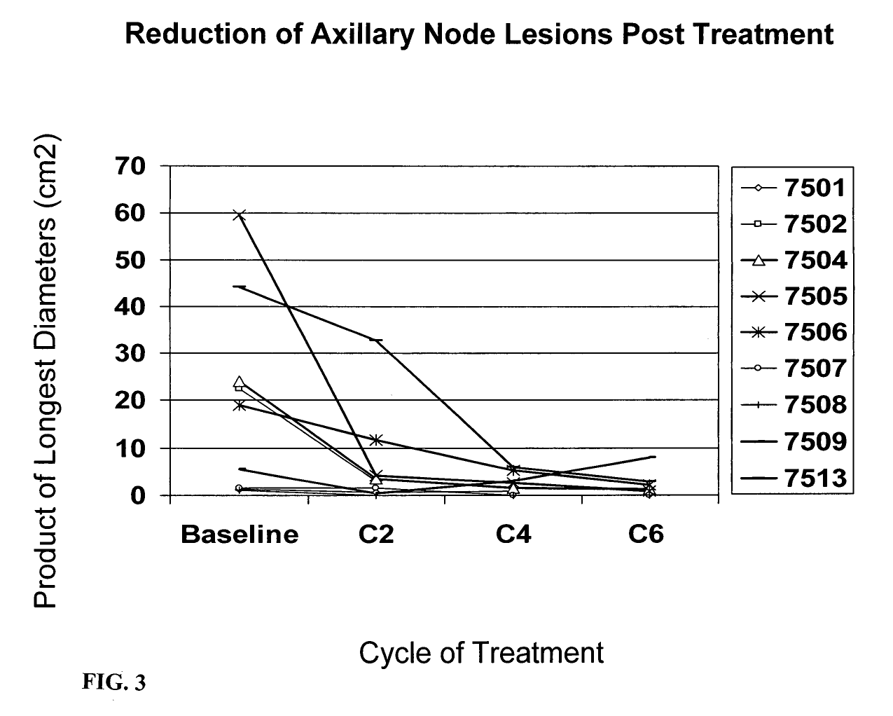

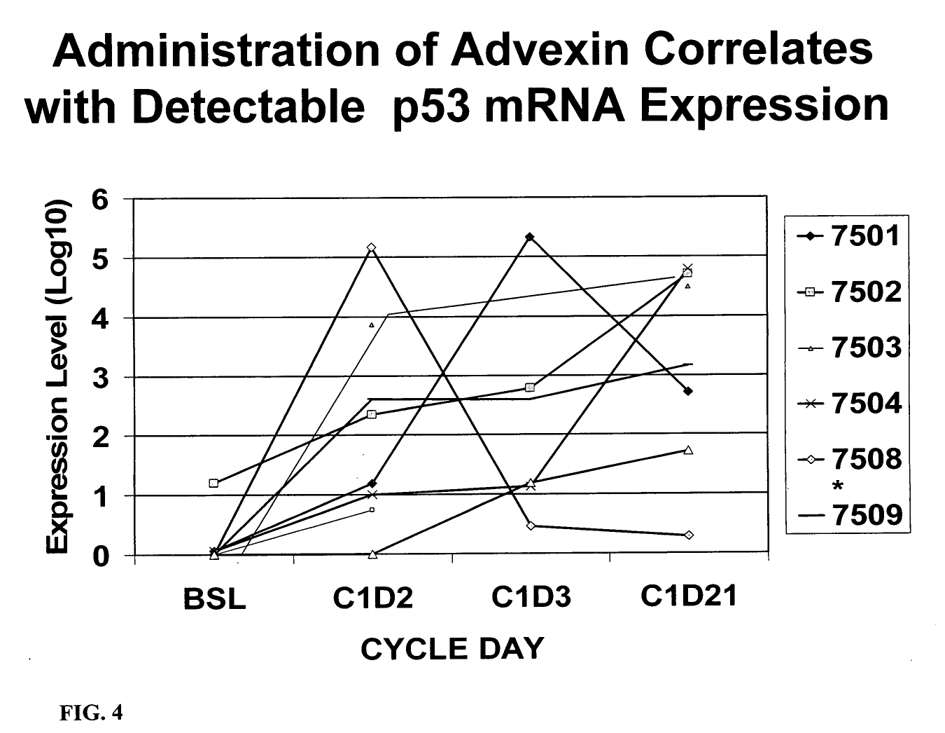

FIG. 2 is a graph showing the reduction in size of primary lesions of various patients, post treatment. FIG. 3 is a graph showing the reduction in size of axillary node lesions of various patients, post treatment. FIG. 4 is a graph showing that administration of Advexin® correlates with detectable p53 mRNA expression.

### EXAMPLE 7

### Expression OF p53 mRNA

Table 5 provides data on the median fold increase in p53 mRNA expression in tumor biopsy specimens following Advexin® administration. In the table, the Frequency of p53 mRNA positives and the median fold increase in p53 mRNA are in comparison to baseline p53 mRNA levels.

TABLE 5

| | | | Median fold |
|---|---|---|---|
| | | Frequency of p53 | increase in p53 |
| Day | N | mRNA positive | mRNA |
| 2 | 7 | 100% | 15 |
| 3 | 6 | 100% | 435 |
| 21 | 6 | 100% | 3000 |

### EXAMPLE 8

### Lymphocyte Infiltration

Table 6 provides the results of lymphocyte infiltration measurements. The relative degree of lymphocyte infiltration is provided along with the percentage of infiltrating cells that are T-cells (CD3⁺) or B-cells (CD20⁺).

TABLE 6

| | Lymphocyte | | |
|---|---|---|---|
| Patient | Infiltration | CD3 (%) | CD20 (%) |
| 7501 | +++ | 80 | 20 |
| 7502 | ++ | 70 | 30 |
| 7503 | ++ | 90 | 10 |
| 7504 | ++ | 90 | 10 |
| 7505 | ++ | 70 | 30 |
| 7506 | ++ | 80 | 20 |
| 7507 | ++ | 95 | 5 |
| 7508 | +++ | 70 | 30 |
| 7509 | ++ | 80 | 20 |
| 7510 | ++ | 90 | 10 |
| 7511 | +++ | 70 | 30 |
| 7512 | ++ | 80 | 20 |

### EXAMPLE 9

### Immunohistochemisty of Tumor Sections

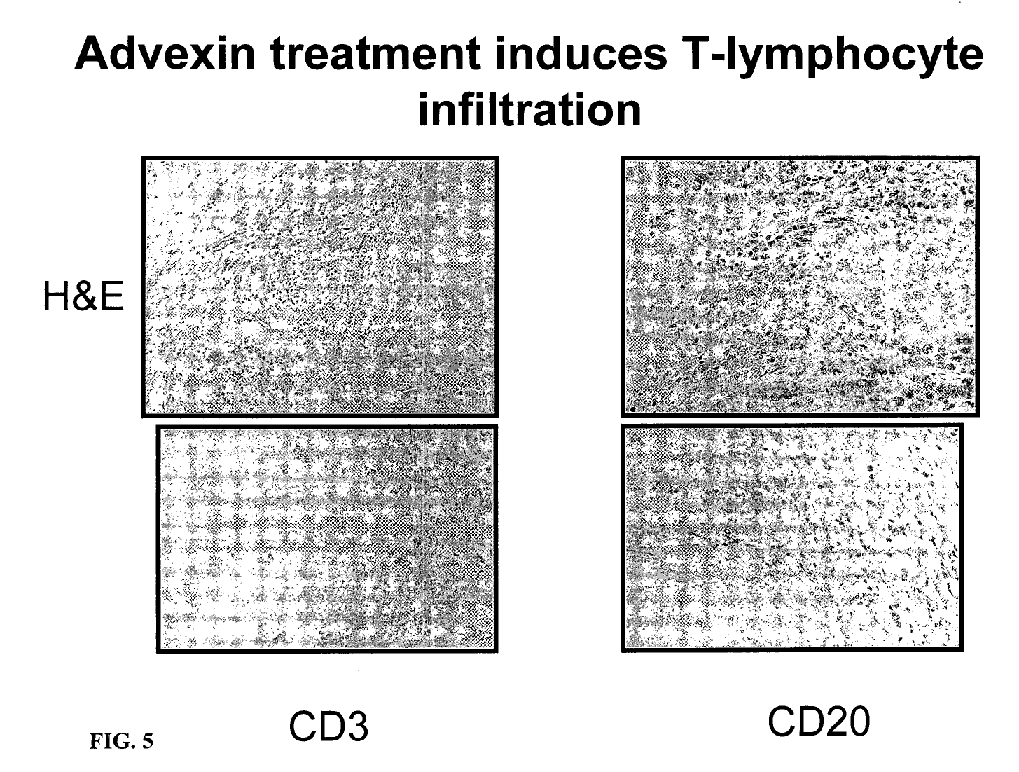

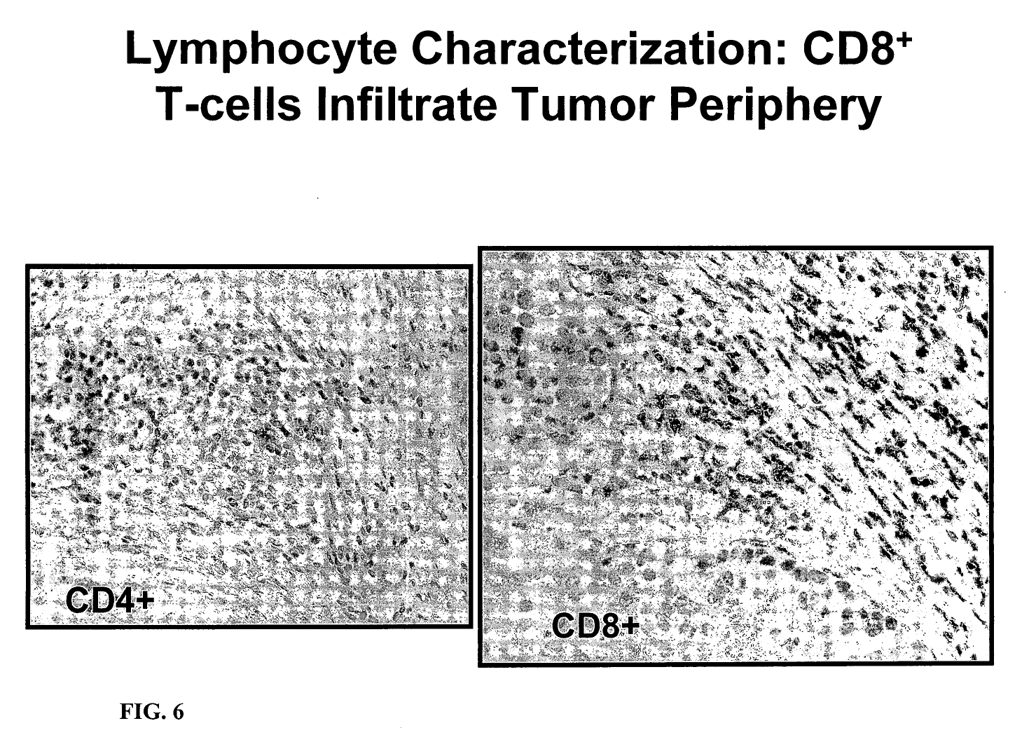

FIG. 5 shows representative tissue sections of tumor biopsy samples stained with hematoxylin and eosin (H&E), and lymphocytes immunostained with antibodies to CD3 and CD20. FIG. 6 shows representative tissue sections of T-cells immunostained with anti-CD4 and anti-CD8 antibodies.

### EXAMPLE 10

### Anti-p53 Antibodies

Table 7 presents the results of measurements of anti-p53 and anti-adenovirus antibodies. In the table, antibody levels for baseline (“BLS”) and Day 21 of the first cycle (“C1D21”) are presented.

TABLE 7

| | |
|---|---|
| Anti p53 | Anti Ad5 |

| Patient | BSL | C1D21 | BSL | C1D21 |
|---|---|---|---|---|
| 7501 | − | − | + | + |
| 7502 | + | + | + | + |
| 7503 | − | − | + | + |
| 7504 | + | + | + | + |
| 7505 | − | − | + | + |
| 7506 | − | − | + | + |
| 7507 | − | − | + | + |
| 7508 | − | − | + | + |
| 7509 | − | − | + | + |
| 7510 | + | + | + | + |
| 7511 | − | − | + | + |
| 7512 | + | + | + | + |

### EXAMPLE 11

### Protocol Synopsis/Summary

Table 8 depicts an exemplary treatment protocol used by the inventors, which can be adapted by those of ordinary skill in the art for treating patients with Advexin® in combination with doxorubicin and docetaxel. The protocol can be adapted for treatment of breast cancer using other chemotherapeutic agents, or treatment of other cancers using one or more chemotherapeutic agents.

TABLE 8

| | |
|---|---|
| TITLE | A phase II, multicenter, single-arm, study of efficacy and safety |
| | of neoadjuvant intra-lesion Advexin ® (Ad5CMV-p53) in |
| | combination with doxorubicin and docetaxel in patients with |
| | non-inflammatory locally advanced breast cancer (LABC). |
| INVESTIGATORS/TRIAL | 3-6 centers in France and USA |
| LOCATION | Centre René Huguenin, Saint-Cloud, France; |
| | Hôspital Saint-Louis, Paris, France; |
| | UT-MD Anderson Cancer Center, Houston TX, USA |
| STUDY OBJECTIVES | |
| Primary: | To determine the therapeutic efficacy of neoadjuvant intratumoral |
| | Advexin ® plus chemotherapy in patients with locally advanced |
| | breast cancer (LABC). |
| Secondary: | To evaluate the safety profile of Advexin ® combined with |
| | doxorubicin and docetaxel chemotherapy regimen. |
| STUDY DESIGN | A multicenter, open label, Phase II trial |
| STUDY POPULATION | Inclusion Criteria: |
| | Female patients with: |
| | Histologically/cytologically proven locally advanced breast |
| | cancer; patients must have measurable disease; |
| | Unilateral T2 N0-2 M0 breast cancer (excluding inflammatory |
| | breast carcinoma); |
| | Tumor able to be treated intra-lesionally with Advexin |
| | No previous immune therapy or chemotherapy; |
| | ECOG (PS) ≦1; |
| | Life expectancy ≧6 months; |
| | Age ≧18 years |
| | Negative serology for HIV 1 and 2, Hepatitis B surface antigen |
| | and Hepatitis C antibody; |
| | Adequate organ function including the following: |
| | 1. WBC count ≧3.0 × 10⁹/L, absolute neutrophils count |
| | (ANC) ≧1.5 × 10⁹/L, platelets ≧100 × 10⁹/L, hemoglobin |
| | ≧10.0 g/dl; |
| | 2. Bilirubin within normal range of institutional value, |
| | aspartate transaminases (AST) or alanine transaminases |
| | (ALT) ≦1.5 times the upper limit of normal (ULN), |
| | alkaline phosphatases ≦3 × ULN; |
| | 3. Kaliaemia, calcaemia and natraemia within normal limits; |
| | 4. Creatinine levels ≦1.5 N or creatinine clearance >60 ml/min; |
| | 5. Left ventricular ejection fraction (LVEF) within normal |
| | limits by echocardiographic or scintigraphic (multiple− |
| | gated acquisition scan) assessment; |
| | Patients with reproductive potential must be using effective |
| | contraceptive methods; |
| | Patients with history of prior invasive malignancies must be |
| | disease-free for at least 5 years prior to study entry; |
| | Adequate tumor tissue (frozen specimen from open biopsy or |
| | core needle aspirate, or paraffin block) must be made available |
| | to determine p-53 status by DNA sequencing (method to be |
| | determined). Patients will be enrolled in the study irrespective of |
| | their original p53 status; |
| | Signed informed consent obtained prior to all study procedures; |
| | negative pregnancy test |
| | Exclusion Criteria: |
| | Her2/neu positive tumor (2+ or 3+) as documented by FISH or |
| | Elisa method; |
| | Multifocal or bilateral or metastatic disease; |
| | No evidence of primary breast lesion (e.g. T0, Tx); |
| | Pregnant or breast-feeding patients; |
| | History of prior malignancies (other than non melanoma skin |
| | cancer or excised cervical carcinoma in situ); |
| | History of atrial or ventricular arrhythmia and/or congestive |
| | heart failure, or second- or third-degree heart block, or history of |
| | clinically and electrocardiographically documented myocardial |
| | infarction; |
| | Other serious illness or medical condition that, in the |
| | investigator's opinion, constitute a contraindication for the |
| | planned treatment or would not permit patient's compliance, i.e. |
| | uncontrolled active infection or psychiatric disorder; |
| | Concurrent treatment with any other experimental drugs or |
| | participation in another clinical trial within 30 days prior to |
| | study screening; |
| | Concurrent treatment with any other anti-cancer therapy; |
| | Prior gene therapy using adenoviral vector(s) or p-53 gene |
| | product; |
| | Known hypersensitivity to study drugs or excipients; |
| | Symptomatic peripheral neuropathy >NCI-CTCAE grade 1; |
| Total expected number of subjects | 34 patients will be included in the first step with 40 additional |
| | patients in the second one for a total of 74 patients. |
| | Two different patient populations have to be entered simultaneously |
| | according to their p-53 status: the first patient category will include |
| | patients with p-53 dysfunction, and the second one will include |
| | patients with normal p-53 gene. |
| | At the first step, 17 evaluable patients will be entered in each patient |
| | category. If at least 4 pCR are observed, 20 additional patients will |
| | be entered, for a total of 37 in each patient category Define |
| | thickness of serial sections. |
| | Patients not evaluable for pathological response will be replaced |
| STUDY DRUGS | |
| Administration route and dose | Advexin ®: An adenoviral vector containing the wild type p-53 |
| | gene with transgene expression driven by the CMV |
| | promoter, will be administered Intratumorally on |
| | days 0 and 1 of each 21-day cycle with fixed dose at |
| | 2.5 × 10¹² viral particles (vp) per injection (and |
| | 1.0 × 10¹² vp radially around the primary lesion?), |
| | followed by |
| | Doxorubicin: 50 mg/m² IV in 15 min, day 1. |
| | Docetaxel: 75 mg/m² IV in 1 hour, immediately after |
| | doxorubicin. |
| | Six cycles of study treatment will be administered. |
| | Patients will undergo complete resection of primary tumor with |
| | node dissection within 6 weeks of last chemotherapy |
| Prophylactic Treatment | All patients should be premedicated with oral corticosteroids such |
| | as dexamethasone 16 mg/day for 3 days starting one day prior to |
| | docetaxel infusion. |
| | Perfilgrastim (Neulasta ®) 6 mg s/c will be administered on day 3 of |
| | each cycle. |
| EVALUATION CRITERIA | |
| Primary Endpoint: | Rate of pathological complete response at surgery |
| | Patients will be considered evaluable for pathological response if |
| | they receive 6 cycles of study treatment and undergo surgery |
| | pCR is defined as disappearance of all tumor with the exception of |
| | in situ carcinoma |
| Secondary Endpoints: | Efficacy: |
| | Rate of objective response according to RECIST |
| | Disease-free interval |
| | Down-staging |
| | Extent of residual loco-regional disease |
| | Safety: |
| | Incidence and severity of adverse events and laboratory |
| | abnormalities (NCI-CTCAE version 3) |
| | Incidence of Serious Adverse Events and treatment |
| | discontinuations |
| | All patients who receive any study treatment will be considered |
| | evaluable for safety |
| | Safety will be evaluated from the time of first administration of |
| | study drugs to 30 days after surgery. |
| STATISTICAL | Two different patient populations have to be entered simultaneously |
| CONSIDERATIONS | according to their p-53 status: the first patient category will include |
| | patients with p-53 dysfunction, and the second one will include |
| | patients with normal p-53 gene. |
| | A Simon two stage design is employed separately for the 2 patient |
| | categories. Assuming that the study regimen will yield a pCR rate |
| | of 40% and that 20% is an uninteresting rate of response, a total of |
| | 37 patients are necessary to obtain an α-risk of 0.05 and β-risk of |
| | 0.15 in each category. |
| | In each category, 17 evaluable patients will be entered in the first |
| | step; if 3 or fewer patients experience pCR in a given category, the |
| | study treatment will be considered insufficiently interesting and |
| | enrollment will be discontinued. If 4 or more responses are |
| | observed, an additional 20 patients will be entered. If at least 12 |
| | evaluable patients in the total 37 patients experience a pCR in a |
| | given patient category, the regimen will be considered sufficiently |
| | interesting for further evaluation. |
| DURATION OF TREATMENT | Patients will receive 6 cycles of treatment, except in the event of |
| | disease progression, unacceptable toxicity, withdrawal of consent |
| | by the patient or investigator decision |

TABLE 9
Schedule of Assessments

| | Screening (N days | | | | | | | End of | |
|---|---|---|---|---|---|---|---|---|---|
| | before treatment | | | | | | | Study | Follow- |
| | initiation) | | | | | | | (28 days | up |
| | | 14 | 7 | Day 1 of each | | Every 2 | | after | (every 3 |
| Procedure | 28 days | days | days | cycle | Weekly | cycles | Surgery | surgery) | months) |
| Informed Consent | | X | | | | | | | |
| Eligibility criteria | | X | | | | | | | |
| Demography | | X | | | | | | | |
| Medical history | | X | | | | | | | |
| Tumor biopsy | | X | | | | | | | |
| Pregnancy test | | X | | | | | | | |
| Physical examination | | X | | X | | | | X | |
| Vital signs | | X | | X | | | | X | |
| Performance Status | | X | | X | | | | X | |
| (WHO) | | | | | | | | | |
| Chest X-ray | | X | | | | X | | X | |
| 12-lead ECG | | | X | | | | | X | |
| LVEF (MUGA) | | | X | | | | | X | |
| Disease assessment | X | | | | | X | | | X |
| (RECIST) | | | | | | | | | |
| Tumor marker | | X | | X | | | | X | X |
| assessment | | | | | | | | | |
| Pathological | | | | | | | X | | |
| assessment | | | | | | | | | |
| Hematology^{a} | | | X | X | X | | | X | |
| Biochemistry^{b} | | | X | X | X | | | X | |
| Adverse events | | | | X | | | | X | X^{c} |
| Concomitant | | X | | X | | | | X | |
| medication | | | | | | | | | |

^{a}Hemoglobin, differential white blood cell count, platelet count
^{b}ALT/SGPT, AST/SGOT, GGT, AP, total bilirubin, sodium, potassium, calcium, magnesium, urea, albumin, creatinine, creatinine clearance (calculated using the Cockroft formula).
^{c}All treatment related Adverse Events should be followed until resolution.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by this application.

### REFERENCES

The following references, to the extent that they provide exemplary procedural or other details supplementary to those set forth herein, are specifically incorporated herein by reference:
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,659,774
U.S. Pat. No. 4,682,195
U.S. Pat. No. 4,683,202
U.S. Pat. No. 4,683,202
U.S. Pat. No. 4,797,368
U.S. Pat. No. 4,816,571
U.S. Pat. No. 4,959,463
U.S. Pat. No. 5,139,941
U.S. Pat. No. 5,141,813
U.S. Pat. No. 5,264,566
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,670,488
U.S. Pat. No. 5,677,178)
U.S. Pat. No. 5,705,629
U.S. Pat. No. 5,739,169
U.S. Pat. No. 5,739,169
U.S. Pat. No. 5,747,469
U.S. Pat. No. 5,747,869
U.S. Pat. No. 5,801,005
U.S. Pat. No. 5,801,005
U.S. Pat. No. 5,824,311
U.S. Pat. No. 5,824,311
U.S. Pat. No. 5,830,880
U.S. Pat. No. 5,830,880
U.S. Pat. No. 5,846,945
U.S. Pat. No. 5,846,945
U.S. Pat. No. 5,928,906
U.S. Pat. No. 5,932,210
U.S. Pat. No. 5,935,819
U.S. Pat. No. 5,981,225
U.S. Pat. No. 5,994,136
U.S. Pat. No. 5,994,136
U.S. Pat. No. 6,013,516
U.S. Pat. No. 6,017,524
U.S. Pat. No. 6,069,134
U.S. Pat. No. 6,136,594
U.S. Pat. No. 6,143,290
U.S. Pat. No. 6,143,290
U.S. Pat. No. 6,210,939
U.S. Pat. No. 6,296,845
U.S. Pat. No. 6,410,010
U.S. Pat. No. 6,410,010
U.S. Pat. No. 6,511,184
U.S. Pat. No. 6,511,847
U.S. Pat. No. 6,627,190
U.S. patent Ser. No. 09/351,778
U.S. Pub. No. 2002/0006914
U.S. Pub. No. 2002/0028785
U.S. Pub. No. 2002/0077313
U.S. Pub. No. 2003/0045499
U.S. Pub. No. 2004/0213764
Abbondanzo et al., Breast Cancer Res. Treat., 16:182(#151), 1990.
Airoldi et al., J. Immunol. 165, 6880-6888 (2000).
Aksentijevich et al., Hum. Gene Ther., 7(9):1111-1122, 1996.
Alam and Cook, Anal. Biochem., 188:245-254, 1990.
Allred et al., Breast Cancer Res. Treat., 16:182(#149), 1990.
Angel et al., Mol. Cell. Biol., 7:2256, 1987.
Atchison and Perry, Cell, 46:253, 1986.
Atchison and Perry, Cell, 48:121, 1987.
Austin-Ward, Villaseca, Rev. Med. Chil., 126(7):838-45, 1998.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bakhshi et al., Cell, 41(3):899-906, 1985.
Banerji et al., Cell, 27(2 Pt 1):299-308, 1981.
Banerji et al., Cell, 33(3):729-740, 1983.
Bargonetti et al., Cell, 65(6):1083-1091, 1991.
Berkhout et al., Cell, 59:273-282, 1989.
Bertholet et al., Eur. J. Immunol., 27(3):798-801 1997.
Blomer et al., J. Virol., 71(9):6641-6649, 1997.
Bodine and Ley, EMBO J, 6:2997, 1987.
Boshart et al., Cell, 41:521, 1985.
Bosze et al., EMBO J, 5(7):1615-1623, 1986.
Braddock et al., Cell, 58:269, 1989.
Brizel, Semin. Radiat. Oncol., 8(4):237-246, 1998.
Brown et al. Breast Cancer Res. Treat., 16:192(#191), 1990.
Bukowski et al., Clin. Cancer Res., 4(10):2337-47, 1998.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chang et al., Mol. Cell. Biol., 9:2153, 1989.
Choi et al., Cell, 53:519, 1988.
Christodoulides et al., Microbiology, 144(Pt 11):3027-37, 1998.
Ciernik et al., Clin. Cancer Res., 2(5):877-882, 1996.
Clayman et al. 1995b
Clayman et al J. Clin. Oncol., 16(6):2221-2232, 1998.
Clayman et al., Cancer Res., 55(14):1-6, 1995.
Cleary and Sklar, Proc. Natl. Acad. Sci. USA, (21):7439-7443, 1985.
Cleary et al., J. Exp. Med., 164(1):315-320, 1986.
Cohen et al., J. Cell. Physiol., 5:75, 1987.
Costa et al., Mol. Cell. Biol., 8:81, 1988.
Cotten et al., Proc. Natl. Acad. Sci. USA, 89(13):6094-6098, 1992.
Couch et al., Am. Rev. Resp. Dis., 88:394-403, 1963.
Coupar et al., Gene, 68:1-10, 1988.
Cripe et al., EMBO J, 6:3745, 1987.
Culotta and Hamer, Mol. Cell. Biol., 9:1376, 1989.
Curiel, Nat. Immun., 13(2-3):141-164, 1994.
Curran, Seminars Radiation Oncol., 8(4Suppl):2-4, 1998.
Dandolo et al., J. Virology, 47:55-64, 1983.
Davidson et al., J. Immunother., 21(5):389-98, 1998.
De Villiers et al., Nature, 312(5991):242-246, 1984.
DeLeo, Crit. Rev. Immunol., 18(1-2):29-35, 1998.
Deschamps et al., Science, 230:1174-1177, 1985.
Dillman Cancer Biother. Radiopharm., 14:5-10, 1999.
Edbrooke et al., Mol. Cell. Biol., 9:1908, 1989.
Edlund et al., Science, 230:912-916, 1985.
el-Kareh and Secomb, Crit. Rev. Biomed. Eng., 25(6):503-571, 1997.
EP 266 032
Feigner et al., Proc. Natl. Acad. Sci. USA, 84(21):7413-7417, 1987.
Feng and Holland, Nature, 334:6178, 1988.
Fields and Jang, Science, 249(4972): 1046-1049, 1990.
Firak and Subramanian, Mol. Cell. Biol., 6:3667, 1986.
Foecking and Hofstetter, Gene, 45(1):101-105, 1986.
Fraley et al., Proc. Natl. Acad. Sci. USA, 76:3348-3352, 1979.
Friedmann, Science, 244:1275-1281, 1989.
Froehler et al., Nucleic Acids Res., 14(13):5399-5407, 1986.
Fujita et al., Cell, 49:357, 1987.
Gabizon et al., Cancer Res., 50(19):6371-6378, 1990.
Gabrilovich et al., J. Immunother. Emphasis Tumor Immunol., 119(6):414-418, 1996.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Graham and Prevec, Biotechnology, 20:363-390, 1992.
Graham et al, J. General Virology, 36:59-74, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Hadden, Int. J. Immunopharmacol., 21:79-101, 1999.
Hanibuchi et al., Int. J. Cancer, 78(4):480-5, 1998.
Hauber and Cullen, J. Virology, 62:673, 1988.
Hellstrand et al., Acta. Oncol., 37(4):347-53, 1998.
Hen et al., Nature, 321:249, 1986.
Herr and Clarke, Cell, 45:461, 1986.
Herz and Gerard, Proc. Natl. Acad. Sci. USA, 90:2812-2816, 1993.
Hirochika et al., J. Virol., 61:2599, 1987.
Hirsch et al., Mol. Cell. Biol., 10:1959, 1990.
Holbrook et al., Virology, 157:211, 1987.
Hollstein et al., Science, 253(5015):49-53, 1991.
Horlick and Benfield, Mol. Cell. Biol., 9:2396, 1989.
Horwich et al. J. Virol., 64:642-650, 1990.
Houbiers et al., Eur. J. Immunol., 23(9):2072-2077, 1993.
Hui and Hashimoto, Infect. Immun., 66(11):5329-36, 1998.
Hurpin et al., Vaccine, 16(2-3):208-215.1998.
Hwang et al., Mol. Cell. Biol., 10:585, 1990.
Imbra and Karin, Nature, 323:555, 1986.
Imler et al., Mol. Cell. Biol., 7:2558, 1987.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Ju et al., Gene Ther., 7(4):329-38, 2000.
Kadesch and Berg, Mol. Cell. Biol., 6:2593, 1986.
Kaneda et al., Science, 243:375-378, 1989.
Karin et al., Mol. Cell. Biol., 7:606, 1987.
Katinka et al., Cell, 20:393, 1980.
Kato et al, J. Biol. Chem., 266:3361-3364, 1991.
Kawamoto et al., Mol. Cell. Biol., 8:267, 1988.
Kelleher and Vos, Biotechniques, 17(6):1110-7, 1994.
Kerr et al., Br. J. Cancer, 26(4):239-257, 1972.
Kiledjian et al., Mol. Cell. Biol., 8:145, 1988.
Klamut et al., Mol. Cell. Biol., 10:193, 1990.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Larsen et al., Proc Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Levinson et al., Nature, 295:79, 1982.
Levrero et al., Gene, 101:195-202, 1991.
Lin et al., Mol. Cell. Biol., 10:850, 1990.
Liu et al., Cancer Res., 55(14):3117-3122, 1995.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Mann et al., Cell, 33:153-159, 1983.
Martin et al., Nature, 345(6277):739-743, 1990.
Mayordomo et al., J. Exp. Med., 183(4):1357-1365, 1996.
McLaughlin et al., J. Virol., 62(6):1963-1973, 1988.
Mercer, Critic. Rev. Eukar. Gene Express. 2:251-263, 1992.
Mietz et al., EMBO J., 11(13):5013-5020, 1992.
Miksicek et al., Cell, 46:203, 1986.
Miller et al., Am. J. Clin. Oncol., 15(3):216-221, 1992.
Moreau et al., Nucl. Acids Res., 9:6047, 1981.
Muesing et al., Cell, 48:691, 1987.
Muzyczka, Curr. Topics Microbiol. Immunol., 158:97-129, 1992.
Naldini et al., Science, 272(5259):263-267, 1996.
Ng et al., Nuc. Acids Res., 17:601, 1989.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Paskind et al., Virology, 67:242-248, 1975.
PCT Appln. PCT/US99/05781
PCT Appln. WO 98/07408
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Picard and Schaffner, Nature, 307:83, 1984.
Pietras et al., Oncogene, 17(17):2235-49, 1998.
Pinkert et al., Genes and Dev., 1:268, 1987.
Porton et al., Mol. Cell. Biol., 10:1076, 1990.
Qin et al., Proc. Nat'l Acad. Sci. USA, 95(24):1411-6, 1998.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Rodriguez and Denhardt (Eds.), Stoneham:Butterworth, 467-492, 1988.
Ripe et al., Mol. Cell. Biol., 9:2224, 1989.
Rosen et al., Cell, 41:813, 1988.
Rosenberg, Nature, 411:380-384, 2001.
Rosenfeld et al., Science, 252:431-434, 1991.
Rosenfeld, et al., Cell, 68:143-155, 1992.
Roth et al., Nat Med., 2(9):985-991, 1996.
Roux et al., Proc. Natl. Acad. Sci. USA, 86:9079-9083, 1989.
Sambrook et al., In: Molecular cloning, Cold Spring Harbor Laboratory Press, Cold Spring Harbor, N.Y., 2001.
Sambrook et al., In: Molecular cloning, Cold Spring Harbor Laboratory Press, Cold Spring Harbor, N.Y., 1989.
Satake et al., J. Virology, 62:970, 1988.
Schaffner et al., J. Mol. Biol., 201:81, 1988.
Sharp and Marciniak, Cell, 59:229, 1989.
Shaul and Ben-Levy, EMBO J, 6:1913, 1987.
Shaw et al., Proc. Natl. Acad. Sci. USA, 89(10):4495-4499, 1992.
Sherman et al., Mol. Cell. Biol., 9:50, 1989.
Sleigh and Lockett, J. EMBO, 4:3831, 1985.
Solodin et al., Biochemistry, 34(41):13537-13544, 1995.
Spalholz et al., Cell, 42:183, 1985.
Spandau and Lee, J. Virology, 62:427, 1988.
Spandidos and Wilkie, EMBO J, 2:1193, 1983.
Stephens and Hentschel, Biochem. J, 248:1, 1987.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Temin, In: Gene Transfer, Kucherlapati (Ed.), NY, Plenum Press, 149-188, 1986.
Thierry et al., Proc. Natl. Acad. Sci. USA, 92(21):9742-9746, 1995.
Thiesen et al., J. Virology, 62:614, 1988.
Top et al., J. Infect. Dis., 124:155-160, 1971.
Topalian et al., J. Immunol., 142:3714-3725, 1989.
Tratschin et al., Mol. Cell. Biol., 4:2072-2081, 1984.
Treisman, Cell, 42:889, 1985.
Tronche et al., Mol. Biol. Med., 7:173, 1990.
Trudel and Constantini, Genes and Dev., 6:954, 1987.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Tyndell et al., Nuc. Acids. Res., 9:6231, 1981.
U.S. Pat. No. 5,925,565
Vannice and Levinson, J. Virology, 62:1305, 1988.
Vasseur et al., Proc Natl. Acad. Sci. USA, 77:1068, 1980.
Wang and Calame, Cell, 47:241, 1986.
Weber et al., Cell, 36:983, 1984.
Weinberg, Science, 254(5035):1138-1146, 1991.
Weinberger et al. Mol. Cell. Biol., 8:988, 1984.
Wilcock and Lane, Nature, 349(6308):429-431, 1991.
Wilder et al., Cancer Res., 59:410-413, 1999a.
Wilder et al., Gene Therapy, 6:57-62, 1999b.
Winoto and Baltimore, Cell, 59:649, 1989.
Wong et al., Gene, 10:87-94, 1980.
Yang and Huang, Gene Therapy, 4 (9):950-960, 1997.
Yanuck et al., Cancer Res., 53(14):3257-3261, 1993.
Yonish-Rouach et al., Nature, 352(6333):345-347, 1991.
Yutzey et al. Mol. Cell. Biol., 9:1397, 1989.
Zakut-Houri et al., EMBO J., 4(5):1251-1255, 1985.
Zhu et al., Science, 261(5118):209-211, 1993.
Zufferey et al., Nat. Biotechnol., 15(9):871-875, 1997.

What is claimed is:

1. A method for inducing an immune response in a tumor in an immunocompetent subject, comprising injecting a first expression construct comprising a nucleic acid segment encoding p53 into the tumor in an amount effective to induce an immune response in the tumor.

2. The method of claim 1, wherein the immune response comprises T-cell lymphocyte infiltration into the tumor.

3. The method of claim 1, wherein the subject is a human.

4. The method of claim 1, wherein the tumor is a cancer.

5. The method of claim 4, wherein the cancer is selected from the group consisting of brain cancer, head & neck cancer, lung cancer, breast cancer, cervical cancer, bladder cancer, skin cancer, and rectal cancer.

6. The method of claim 5, wherein the cancer is breast cancer.

7. The method of claim 1, further comprising detecting an immune response in the tumor.

8. The method of claim 7, wherein detecting an immune response comprises detecting an increase in tumor size within one week following injection.

9. The method of claim 8, wherein detecting an increase in tumor size is performed by palpation of the tumor or by imaging of the tumor.

10. The method of claim 9, wherein imaging of the tumor is by CT or MRI.

11. The method of claim 7, further comprising performing a biopsy of the tumor or surgically excising the tumor following injection of the first expression construct into the tumor.

12. The method of claim 11, comprising detecting T-cells in the tumor, measuring T cell specific proteins in the tumor, and/or measuring T cell specific nucleic acids in the tumor tissue.

13. The method of claim 1, wherein the first expression construct is injected more than one time into the tumor.

14. The method of claim 1, further comprising injecting a second expression construct comprising a nucleic acid segment encoding p53 into the tumor, wherein the second expression construct is not the same as the first expression construct.

15. The method of claim 14, wherein the second expression construct is injected after detecting an immune response in the tumor following injection of the first expression construct.

16. The method of claim 1, wherein the nucleic acid segment encoding p53 is under the control of a promoter active in the cells of the tumor.

17. The method of claim 16, wherein the promoter is CMV IE, RSV LTR, β-actin, Ad-E1, Ad-E2 or Ad-MLP.

20. The method of claim 1, wherein the first expression construct is injected intra-tumorally, to tumor vasculature, or local to a tumor.

21. The method of claim 1, wherein the first expression construct is a viral expression construct.

22. The method of claim 1, further comprising identifying a subject.
